(19) United States
(12) Patent Application Publication
(10) Pub. No.: US 2026/0298372 A1
(43) Pub. Date: Oct. 1, 2026

(54) METHOD AND SYSTEM FOR ASSEMBLING A SYSTEM INCLUDING A FLEXIBLE TUBE

(71) Applicant: Richard Charles Raczuk, Lake Havasu City, AZ (US)

(72) Inventor: Richard Charles Raczuk, Lake Havasu City, AZ (US)

(21) Appl. No.: 19/361,882

(22) Filed: Oct. 17, 2025

Related U.S. Application Data
(60) Provisional application No. 63/708,505, filed on Oct. 17, 2024.

(51) Int. Cl. F16L 3/00; A61M 16/08

(52) U.S. Cl. CPC F16L 3/003; A61M 16/0816

(57) ABSTRACT

A tool system and method for application in the assembly/disassembly of a flexible corrugated tube/hose with another tube/hose and/or component.

The present application relates generally to a method and system for engaging a flexible tube with another item such as a fitting and/or another tube. More specifically, the present application relates to assembling/disassembling a corrugated flexible tube/hose with a fitting or another tube/hose in a medical application.

The present application addresses prior issues with assembling/disassembling flexible tubes/hoses with other components to alleviate the deforming and/or collapsing of the tube/hose in a novel and non obvious manner..

### SUMMARY

One embodiment of the present application contemplates a method for coupling a corrugated flexible hose with component, comprising: placing a portion of the corrugated flexible hose on a first tool portion configured to receive the hose; engaging a rib of the corrugated flexible hose within an engagement portion of the first tool portion; placing a second tool portion configured to receive the hose on the corrugated flexible hose to abut with the first tool portion, wherein the engagement portion of the second tool portion aligns with the engagement portion of the first tool portion and the rib; holding the first and second tool portion together around the corrugated flexible hose to restrain movement of the hose relative to the first and second tool portion; and moving the corrugated flexible hose and component relative to one another io connect the hose and component together.

Other embodiments include apparatuses, systems and kits associated with assembly and disassembly of a corrugated flexible tube/hose with another hose and/or component. Further embodiments, forms, features, aspects, benefits, and advantages of the present application shall become apparent from the description and figures provided herewith.

### BRIEF DESCRIPTION OF THE FIGURES

FIG. 1 depicts one form of flexible corrugated tube/hose to be connected with a fitting component.

FIG. 2 depicts the flexible corrugated tube/hose and fitting of FIG. 1 after being connected.

FIG. 3 depicts an embodiment of the tool having a portion configured with the flexible corrugated hose disposed therein.

FIG. 4 depicts an embodiment of the tool of FIG. 3.

FIG. 5 depicts an enlarged view of a portion of the tool of FIG. 3.

FIG. 6 depicts an exemplary embodiment of mating flexible corrugated tube/hoses with a component and the tools.

FIG. 7 is an illustrative view of one embodiment of the tool system of the present application integrated with a mechanical press.

### DETAILED DESCRIPTION OF THE ILLUSTRATIVE EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

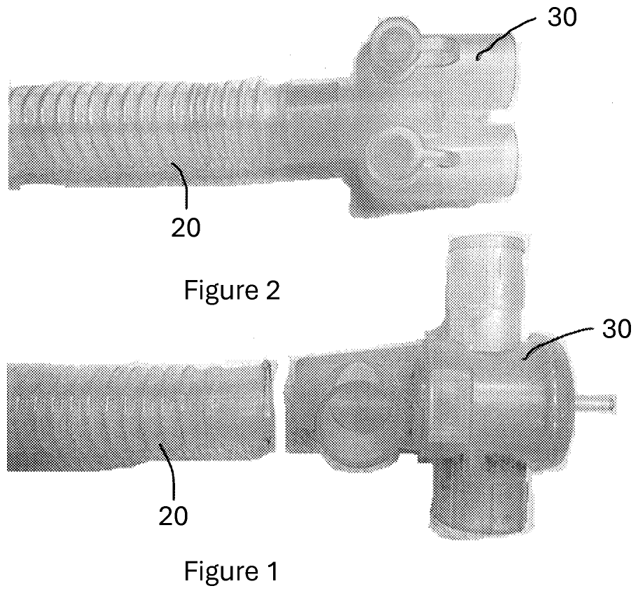

With reference to FIG. 1, there is illustrated an example of a flexible corrugated tube/hose 20 and an end fitting 30 in a disassembled state. With reference to FIG. 2, there is illustrated the corrugated tube/hose 20 coupled with the end fitting 30. The present application addresses prior issues with assembling/disassembling flexible tubes/hoses with other components to alleviate the deforming and/or collapsing of the tube/hose 20. The present application is particularly useful in addressing flexible tubes/hoses utilized in the medical industry.

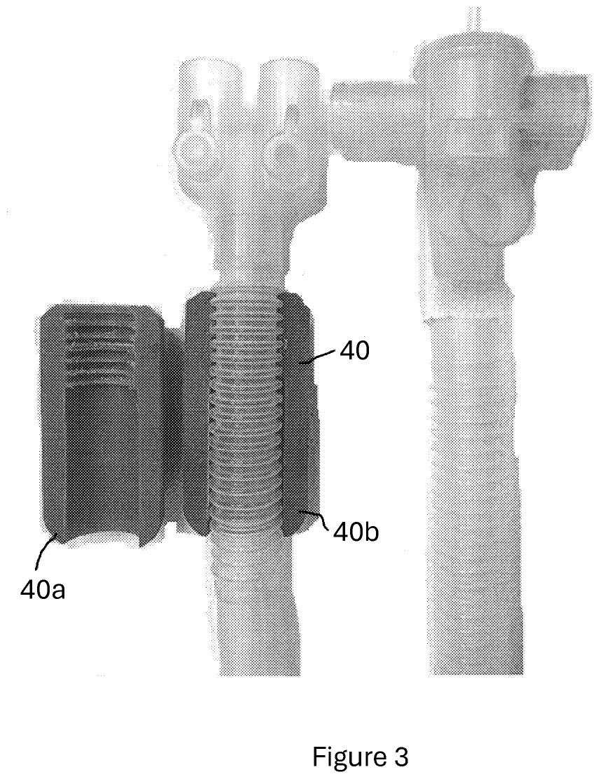

With reference to FIG. 3, there is illustrated one embodiment of the tool 40, having mating portions 40a and 40b for engaging the tube/hose 20. In one embodiment the portions 40a and 40b are located to surround a section of the tube/hose 20. The portions 40a and 40b are configured to be engageable and hold the tube/hose during the assembly/disassembly. In a preferred form the portions include a plurality of engagement ridges that are defined by walls 51.

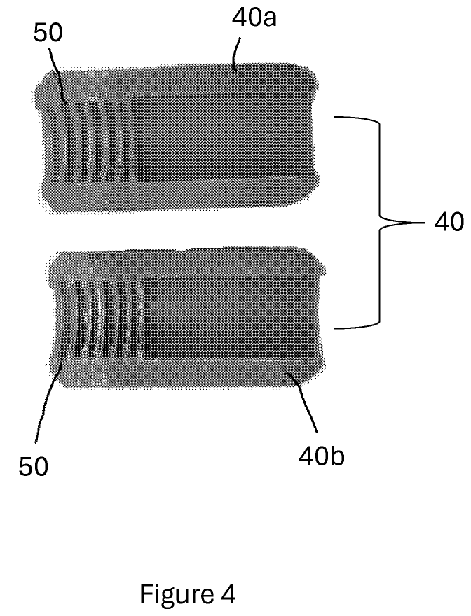

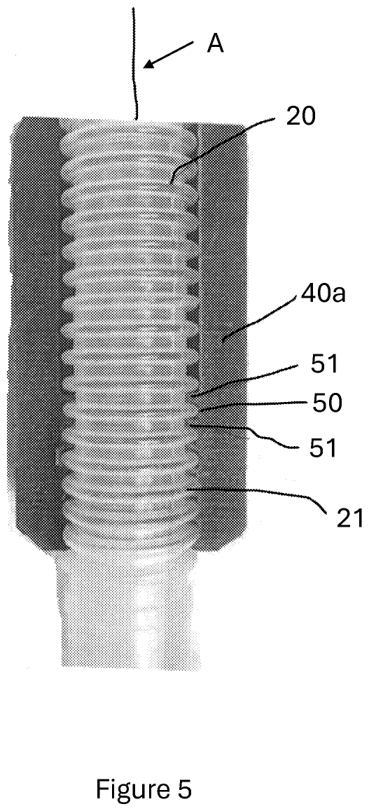

With reference to FIGS. 4 and 5, there is illustrated one embodiment of the tool 40. The tool 40 includes groves 50 that are separated by walls 51. The walls 51 and grooves 50 are sized and spaced to engage at least one of the upstanding ridges 21 formed in the corrugated tube/hose 20. The present figures illustrate multiple engagement with the upstanding ridges, however other embodiments contemplate one or more engagements. In a preferred form a the tool includes portions 40a and 40b each having a plurality of axially spaced grooves that are configured to receive a portion of the tube/hoe 20. IN one embodiment the grooves are uniformly spaced to match the spacing of the tub/hose 20. Other embodiments of the present application contemplate that the grooves 50 are axially spaced apart to receive the tube/hose and skip some of raised ribs on the tube/hose 20. More specifically, the present application includes forms where some of the raised ribs on the tube/hose 20 are received within grooves 50 of the portions 40a and 40b and some of the raised ribs adjacent to those ribs received within grooves are not captured by a groove.

The present preferred form of the portions 40a and 40b has the grooves 50 when the hose 20 is located within the tool 40 and closed extending completely around the circumference of the hose 20. In alternate forms of the present application the grooves are contemplated as extending around less than the entire circumference of the hose 20. That is when assembled the tool includes grooves 50 that the hose ribs reside in but are not completely around the circumference of the hose 20.

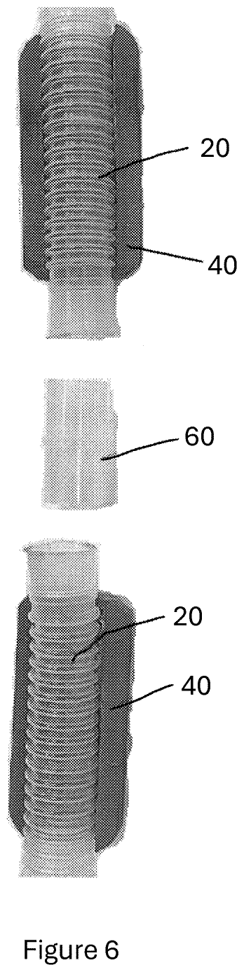

With reference to FIG. 6, there is illustrated the connection of two tubes/hoses 20 together through a coupler 60. Each of the tubes/hoses 20 has a tool 40 engaged with the tubes/hoses 20 to enable assembly/disassembly. As described above each of the tools 40 include a portion 40a and 40b the receive the tube/hose 20 and hold it for assembly or disassembly. Therefore, providing support to prevent the flexing or bending of the hose(s) 20 that make assembly very difficult.

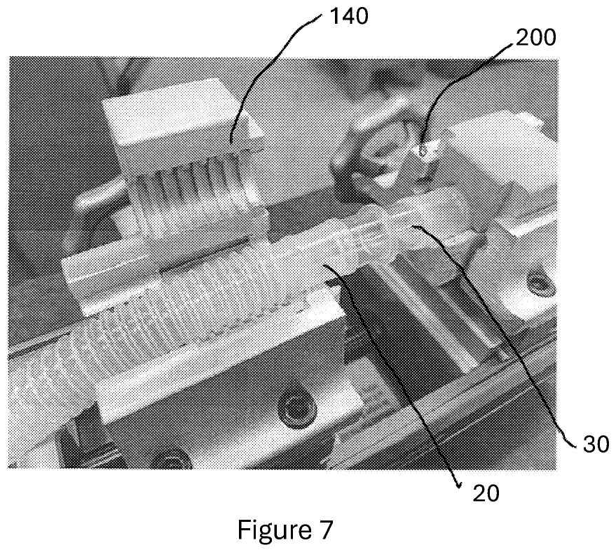

With reference to FIG. 7, there is illustrated another embodiment utilizing a mechanical press 200 with the tool 140 to assemble/disassemble the tube/hose 20 with the end fitting 30. The tool 140 is substantially similar to the prior described tool 40. The press 200 holds the toll 140 in a fixed position with the hose 20 and aligned with the moveable portion of another tool 140 that holds the component to be mated with the hose 20. In the embodiment illustrated a handle is turned to advance the moveable tool 140 on a moveable bed coupled with a lead screw. The present application contemplates that one or both components can be moveable by a variety of mechanical devices.

The present application includes methods for assembling/disassembling the tube/hose 20 with the end fitting 30 or connector 60. The portions of the tool 40 (40a and 40b) are placed around the tube/hose 20 such that at least one of the upstanding ridges 21 is engaged in the grove 50 and the upstanding ridge is prevented from moving relative to the tool 40 in the linear direction indicated by line‘A’. The portions 40a and 40b are held in place to engage the upstanding ridge as the tube/hose 20 and the end fitting 30 or connector 60 are moved relative to one another to connect the components together. The movement can be provided by manual interaction by a person or through a secondary machine to cause the relative movement. To disassemble the process is the same but movement is in the opposite direction.

The present application contemplates the provision of a kit to include appropriate tools 40 in a multitude of sizes for addressing the needs of the person wishing to assemble/disassemble a flexible hose with another hose or component.

While the invention has been illustrated and described in detail in the drawings and foregoing description, the same is to be considered as illustrative and not restrictive in character, it being understood that only the preferred embodiments have been shown and described and that all changes and modifications that come within the spirit of the inventions are desired to be protected. It should be understood that while the use of words such as preferable, preferably, preferred or more preferred utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

What is claimed is:

1. A method for coupling a corrugated flexible hose with component,
comprising:
placing a portion of the corrugated flexible hose on a first tool portion configured to receive the hose;
engaging a rib of the corrugated flexible hose within an engagement portion of the first tool portion;
placing a second tool portion configured to receive the hose on the corrugated flexible hose to abut with the first tool portion, wherein the engagement portion of the second tool portion aligns with the engagement portion of the first tool portion and the rib;
holding the first and second tool portion together around the corrugated flexible hose to restrain movement of the hose relative to the first and second tool portion; and
moving the corrugated flexible hose and component relative to one another io connect the hose and component together.
